SEQUENCE LISTING (1 sequence)

SEQ ID NO: 1 - RNA, 20 nt, Artificial Sequence
  Synthetic
uccauucggcuccaaaccgg

(19) United States
(12) Patent Application Publication
(10) Pub. No.: US 2010/0255112 A1
(43) Pub. Date: Oct. 7, 2010

(54) Efficient Nuclear Delivery of Antisense Oligonucleotides or siRNA In Vitro and In Vivo by Nano-Transforming Polymersomes

(75) Inventors: Dennis E. Discher, Philadelphia, PA (US); Manorama Tewari, East Lansdowne, PA (US); Younghoon Kim, Bethlehem, PA (US)

Correspondence Address: DRINKER BIDDLE & REATH;ATTN: INTELLECTUAL PROPERTY GROUP, ONE LOGAN SQUARE, SUITE 2000, PHILADELPHIA, PA, 19103-6996, US

(73) Assignee: The Trustees of the University of Pennsylvania

(21) Appl. No.: 12/514,784

(22) PCT Filed: Nov. 14, 2007
(86) PCT No.: PCT/US07/23894

§ 371 Date: Jun. 7, 2010

Related U.S. Application Data
(60) Provisional application No. 60/858,862, filed on Nov. 14, 2006.

(51) Int. Cl. A61K 9/50; C12N 5/071; A61K 31/7105; A61P 21/00

(52) U.S. Cl. 424/501; 435/366; 435/325; 424/490; 514/44 A

(57) ABSTRACT

Provided is a biocompatible polyethylene oxide (PEO)-based polymersome system for the delivery of oligonucleotides, including antisense RNA, siRNA and RNAi, to a cell or tissue target, and method of use therefore, wherein the method comprises encapsulating the oligonucleotide in a biodegradable neutral, nano-transforming polymersome delivery vehicle and delivering the encapsulated oligonucleotide to the cell or tissue target in vitro or in vivo, particularly for treating a disease, such cancer or cellular hyperproliferation. The degradable polymersome, and the oligonucleotides stably encapsulated therein are taken up passively by cells and delivered into endolysosomes, wherein the polymersomes decompose at a known rate at a known pH, thereby releasing encapsulated oligonucleotides in a controlled manner within the cell and facilitating delivery of antisense oligonucleotide or siRNA or RNAi into the nucleus of the cell target.

### REFERENCE TO RELATED APPLICATIONS

This patent application claims priority to Provisional Application 60/858,862 filed Nov. 14, 2007, which is herein incorporated in its entirety.

### GOVERNMENT SUPPORT

This work was supported in part by a grant from the National Institutes of Health, Grant No. R21. The government may have certain rights in this invention.

### FIELD OF THE INVENTION

The present invention is related to PEO-based polymersomes and their use as controlled release delivery vehicles for the delivery of nucleic acids, such as antisense oligonucleotides and siRNA, in vitro and in vivo.

### BACKGROUND OF THE INVENTION

Antisense agents range from double-stranded RNA-interference that catalyze mRNA degradation (Fire et al., Nature 391:806-811 (1998)) to single-stranded antisense oligonucleotides (AON) that are finding applications in gene-specific therapies for various diseases. Recent advances in the bio-stability of AONs have been especially significant with 2′O-methyl modifications defining one important class of particularly stable AONs. (Kurreck, Eur. J. Biochem. 270:1628-1644 (2003)) However, stability against degradation does not guarantee functional and efficient delivery, which is still a significant problem with antisense therapies.

The discovery that antisense not only inhibits transcription and/or translation of genes in a sequence-specific manner, but can also splice out exons to circumvent genetic mutations has opened up new modalities for molecular therapy. (Shi et al., J. Control. Release 97:189-209 (2004), Wilton et al., Current Opin. MoL Therapeutics 8:130-5 (2006)). One disease that now appears amenable to AON treatments is Duchenne Muscular Dystrophy (DMD), for which recent cell and animal studies with viruses and various transfection reagents demonstrate AON-induced exon-splicing of the dystrophin transcript. (Lu et al., Nature Medicine 9:1009-1014 (2003), Wells et al., FEBS 552, 145 (2003), Mann et al., PNAS U.S.A. 98:42-27 (2001), Williams et al., Mol. Therapy 14:88-96 (2006), Bremmer-Bout et al., Mol. Therapy 10:232-240 (2004), 't Hoen et al., Pharmacogenomics 7:281-97 (2006)). However, efficient delivery of such AON to muscle is often a significant challenge and much depends on the carriers that provide protection against AON degradation and clearance as well as mechanisms for circulation, cell entry and release into the nucleus. (Dass, Pharmacy and Pharmacology 54:3-27 (2002), Lorenz et al., Mol. Bio. Cell 9:1007-1023 (1998)).

Controlled release polymer vesicles or ‘polymersomes’ with an aqueous lumen for soluble compounds have recently been formulated using either oxidation-sensitive (Napoli et al., Nature Mat. 3:183-189 (2004)) or hydrolysis-sensitive block copolymer amphiphiles. (Ahmed et al., J. Control. Release 96:37-53 (2004), Discher et al., Science 297:967-973 (2002)). Self-porating polymersomes indeed have multiple potential advantages for nucleic acid delivery. Polymer vesicles have already been exploited for nuclear delivery of DNA-intercalating drugs. (Ahmed et al., Molecular Pharmaceutics 3(3):340-350 (2006)

It will be highly desirable to design “stimuli-responsive” vesicles; ones that entrap soluble substance in water, and maintain their stability during the circulation, but become effectively destabilized upon a specific environmental stimulus, to fast release the encapsulants when reaching the target.

Responsive block copolymer self-assemblies that are sensitive to external stimuli, including temperature, pH, electrolyte concentration and electrical potentials are of great interest as novel containers, micro-reactors and actuators to mimic natural systems.

Nano transforming assemblies have attracted much attention because they break down to non-toxic metabolites. They are the key solutions to many environmental problems, and are particularly useful for various biomedical applications. Much work has been focused on degradable polymers and their co-polymers as bulk, or films and monolayers. Only limited work has explored the degradable amphiphilic copolymer self-assemblies (spherical micelles, worm micelles and vesicles) in solutions, which are quite important for soft-material engineering. Mostly spherical micelles, and in rare cases, vesicles, have been reportedly made from copolymers with degradable polyester, typically polylactide or polycaprolactone, as the hydrophobic block, connected to biocompatible, stealthy poly (ethylene oxide) as the hydrophilic block.

Until the present invention, there remained a need in the art for a biocompatible liposome-like delivery system for water-soluble molecules and therapeutics, while retaining stability in circulation in vivo and reducing immuno-response.

### SUMMARY OF THE INVENTION

The present invention provides neutral polymersome vesicles that are both biocompatible and immuno-compatible and capable of encapsulating a molecular composition within the vesicle.

The present invention further provides a method for the controlled delivery of “active agents,” such as molecular compositions, to selected targets by encapsulation of active agents within controlled-release polymersome vehicles.

The polymersomes of the present invention are shown to be able to encapsulate a range of compositions into the membrane cores of the polymersomes. An enormously wide range of hydrophilic materials can be associated with or encapsulated within a polymersome. The present invention, therefore, provides polymersomes which encapsulate one or more “active agents,” which include, without limitation, compositions, such as antisense oligonucleotides, ribozyme molecules, siRNA or RNAi molecules, or fragments thereof, forming a “loaded” or “encapsulated” polymersome.

The present invention further provides methods of using the polymersome to transport one or more selected active agents, such as antisense, ribozyme or RNAi molecular compositions to a patient in need thereof. The polymersomes could be used to deliver active agents to a patient's tissue or blood stream, from which it will ultimately be delivered into the nucleus of individual cells. For example, the polymersomes effectively deliver a therapeutic active agent, such as antisense RNA, to the nucleus of a cell in a patient in need thereof, thus serving as molecular therapy for diseases with underlying molecular basis, such as, but not limited to, cancer.

Provided are stable, synthetic, self-assembling, controlled release, polymersome vesicles, having a semi-permeable, thin walled encapsulating membrane and at least one encapsulant therein, and delivering the encapsulant to the nucleus of a cell in vitro and in vivo. The polymersomes are made by self-assembly in various aqueous solutions of purely synthetic, amphiphilic molecules, such as amphiphilic copolymers. In particular, polyethylene oxide (PEO) based polymersomes of the present invention provide drug delivery vehicles for controlled encapsulation, transportation, and release of encapsulated material.

Additional objects, advantages and novel features of the invention will be set forth in part in the description, examples and figures which follow, and in part will become apparent to those skilled in the art on examination of the following, or may be learned by practice of the invention.

### BRIEF DESCRIPTION OF THE FIGURES

The foregoing summary, as well as the following detailed description of the invention, will be better understood when read in conjunction with the appended drawings. It should be understood, however, that the invention is not limited to the precise arrangements and instrumentalities shown.

FIG. 1 shows the average hydrodynamic size of polymersome vesicles by dynamic light scattering. Polymersome size transitions from vesicles of approximately 100 nm to micelles of approximately 40 nm as controlled-release of encapsulant occur.

FIG. 2 shows release kinetics of antisense oligonucleotide (AON)-encapsulated degradable polymersomes. Release kinetics increases with increasing temperature; at 4° C., leakage is undetectable for days.

FIG. 3 shows hydrodynamic size of PEO-PLA polymersome vesicles with and without encapsulation of material. Encapsulation of siRNA (15 kDa) slightly increases vesicle size.

FIG. 4 shows gene silencing of lamin A/C in vitro. Lamin expression was measured by fluorescence immunoassay after a 72 hour incubation of cells with siRNA-encapsulated polymersomes.

FIG. 5 shows gene silencing of lamin A/C after a 96 hour incubation of cells with siRNA-encapsulated polymersomes.

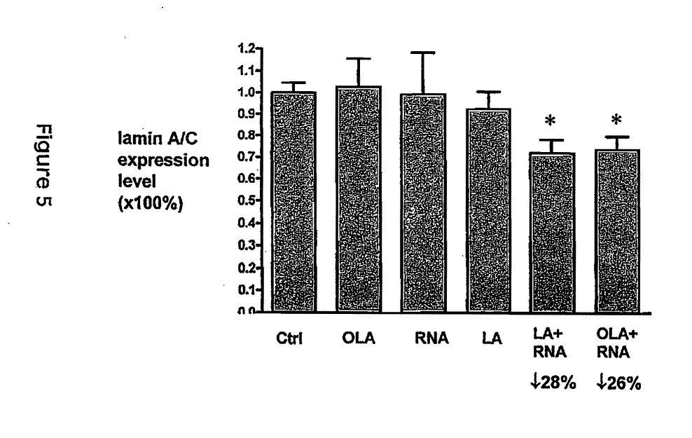

### DETAILED DESCRIPTION OF CERTAIN EMBODIMENTS OF THE INVENTION

The formation of “polymersomes,” stable, yet biodegradable, vesicles comprising large semi-permeable, thin-walled encapsulating membranes, self-assembled in aqueous solutions of amphiphilic block copolymers, has been previously disclosed in published U.S. Patent Application US2005/0003016 and U.S. Pat. Nos. 6,835,394 and 7,217,427, the contents of which are hereby incorporated by reference in their entirety. The polyethylene oxide (PEO)-based polymersome vesicles according to the current invention are, however, unique in that they are neutral, nano-transforming polymersomes capable of encapsulating an active agent, such as an antisense oligonucleotide molecule, for delivery and transport into a cell as well as targeting destabilization of vesicle membrane, thereby facilitating release of encapsulated oligonucleotide in a controlled manner.

Polymersome vesicles. “Polymersomes” of the present invention are synthetic vesicles assembled from amphiphilic block copolymers that offer several material design and performance advantages over vesicles from small molecular weight surfactants and biological lipids. The rational design and synthesis of well-defined block copolymers, having desired molecular weight, volume fraction, and chemistry, improve vesicle stability while retaining the fluidity and deformability similar to that of lipid vesicles. In particular, poly(ethylene oxide) (PEO) based polymersomes of the present invention are robust drug delivery vehicles for the controlled encapsulation, transportation, and release of encapsulated material.

“Vesicles,” as the term is used in the present invention, are essentially semi-permeable bags of aqueous solution as surrounded (without edges) by a self-assembled, stable membrane composed predominantly, by mass, of either amphiphiles or super-amphiphiles which self-assemble in water or aqueous solution.

“Nano-transforming,” as the term is used in the present invention, refers to nano-transforming assemblies comprised of degradable polymeric materials with hydrolysable backbones. The degradable polyester, typically polylactide or polycaprolactone, as the hydrophobic block, can be connected to biocompatible polyethelyne oxide (PEO) as the hydrophilic block. Degradation of the hydrolysable backbones results in changes in morphology of the vesicles. Thus, polymersomes of the present invention are “biodegradable” in that as the vesicles undergo hydrolytic degradation, changes in membrane morphology facilitate the release of materials encapsulated within the membrane.

Polymersomes of the present invention are assembled from synthetic polymers in aqueous solutions. Unlike liposomes, a polymersome does not include lipids or phospholipids as its majority component. Consequently, polymersomes can be thermally, mechanically, and chemically distinct and, in particular, more durable and resilient than the most stable of lipid vesicles. In one exemplary implementation, polymersomes are neutral (as in not exhibiting a positive or negative charge), nano-transforming particles. The polymersomes assemble during processes of lamellar swelling, e.g., by film or bulk rehydration, or through an additional phoresis step, or by other known methods. Like liposomes, polymersomes form by “self assembly,” a spontaneous, entropy-driven process of preparing a closed semi-permeable membrane.

The polymersomes of the present invention are vesicles prepared from diblock amphiphilic copolymers having a molecular weight of greater than a range of 1-4000 g/mol. An “amphiphilic” substance is one containing both polar (water-soluble) and hydrophobic (water-insoluble) groups. “Polymers” are macromolecules comprising connected monomeric heterogeneous molecules. The physical behavior of the polymer is dictated by several features, including the total molecular weight, the composition of the polymer (e.g., the relative concentrations of different monomers), the chemical identity of each monomeric unit and its interaction with a solvent, and the architecture of the polymer (whether it is single chain or branched chains). For example, in polyethylene glycol (PEG), which is a polymer of ethylene oxide (EO), the chain lengths which, when covalently attached to a phospholipid, optimize the circulation life of a liposome, is known to be in the approximate range of 34-114 covalently linked monomers (EO₃₄ to EO₁₁₄).

“Block copolymers” are polymers having at least two, tandem, interconnected regions of differing chemistry. Each region comprises a repeating sequence of monomers. Thus, a “diblock copolymer” comprises two such connected regions (A-B); a “triblock copolymer,” three (A-B-C), etc. Each region may have its own chemical identity and preferences for solvent. Thus, an enormous spectrum of block chemistries is theoretically possible, limited only by the acumen of the synthetic chemist.

The preferred copolymers of the present invention comprise a hydrophilic PEO (polyethylene oxide) block and one of several hydrophobic blocks that drive self-assembly of polymersomes. The diblock or triblock copolymer amphiphiles that mimic the flexibility of various cytoskeletal filaments are described in U.S. Pat. No. 6,835,394, and pending applications related thereto, including U.S. Ser. No. 10/882,816, herein incorporated by reference. The PEO block of the polymer (which is the same as polyethylene-glycol; PEG) is widely known to make interfaces very biocompatible. Thus, the resulting polymersomes are amphiphilic aggregates and fluidity and hydrodynamics play important roles in their formation. The polymersomes are stable in blood in vitro and in blood flow in vivo.

The relevant class of amphiphilic molecules is represented by, but not limited to, block copolymers, e.g., hydrophilic polyethyleneoxide (PEO) linked to hydrophobic polyethylethylene (PEE), or polylactic acid (PLA). The synthetic diversity of block copolymers provides the opportunity to make a wide variety of vesicles with material properties that greatly expand what is currently available from the spectrum of naturally occurring phospholipids.

Table 1 (see Example 1 below) lists some of the synthetic amphiphiles of many kilograms per mole in molecular weight, which are capable of self-assembling into semi-permeable vesicles in aqueous solution. The panel of preferred PEO-PEE block copolymers ranges in molecular weight from 1400 to 8700, with hydrophilic volume fraction, f_{EO}, ranging from 20% to 50%. Table 1 is intended only to be representative of the synthetic amphiphiles suitable for use in the present invention. It is not intended to be limiting. A plethora of molecular variables can be altered with these illustrative polymers, hence a wide variety of material properties are available for the preparation of the polymersomes. One of ordinary skill in the art will readily recognize many other suitable block copolymers that can be used in the preparation of polymersomes based on the teachings of the present invention.

Encapsulated polymersome vesicles. Polymersomes of the present invention are capable of “encapsulating” an active agent within the vesicle membrane, thus polymersomes are encapsulating membranes. Encapsulating membranes, by definition, compartmentalize by being semi- or selectively permeable to solutes, either contained inside or maintained outside of the spatial volume delimited by the membrane. An “encapsulant” in the present invention refers to one or more active agents, such as nucleic acid (RNA/DNA), antisense oligonucleotides (AON), siRNA, RNAi and the like, which are “encapsulated” or “loaded” within the polymersome vesicles for delivery to a cell or tissue target.

By “nucleic acid” or “oligonucleotide” is meant any nucleic acid, whether composed of deoxyribonucleosides or ribonucleosides, and whether composed of phosphodiester linkages or modified linkages, such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages.

It is not intended that the present invention be limited by the nature of the nucleic acid employed. The target nucleic acid may be native or synthesized nucleic acid. The nucleic acid may be from a viral, bacterial, animal or plant source. The nucleic acid may be DNA or RNA and may exist in a double-stranded, single-stranded or partially double-stranded form. Furthermore, the nucleic acid may be found as part of a virus or other macromolecule. See, e.g., Fasbender et al., 1996, J. Biol. Chem. 272:6479-89 (polylysine condensation of DNA in the folin of adenovirus).

Nucleic acids useful in the present invention include, by way of example and not limitation, oligonucleotides and polynucleotides, such as antisense DNAs and/or RNAs; ribozymes; DNA for gene therapy; viral fragments including viral DNA and/or RNA; DNA and/or RNA chimeras; mRNA; plasmids; cosmids; genomic DNA; cDNA; gene fragments; various structural forms of DNA including single-stranded DNA, doublestranded DNA, supercoiled DNA and/or triple-helical DNA; Z-DNA; and the like. The nucleic acids may be prepared by any conventional means typically used to prepare nucleic acids in large quantity. For example, DNAs and RNAs may be chemically synthesized using commercially available reagents and synthesizers by methods that are well-known in the art (see, e.g., Gait, 1985, Oligonucleotide Synthesis: A Practical Approach (IRL Press, Oxford, England)). RNAs may be produce in high yield via in vitro transcription using plasmids, such as SP65 (Promega Corporation, Madison, Wis.).

The nucleic acids may be purified by any suitable means, as are well known in the art. For example, the nucleic acids can be purified by reverse phase or ion exchange HPLC, size exclusion chromatography or gel electrophoresis. Of course, the skilled artisan will recognize that the method of purification will depend in part on the size of the nucleic acid to be purified.

As used herein, the term “antisense oligonucleotide” (AON) means a nucleic acid polymer, at least a portion of which is complementary to a nucleic acid which is present in a normal cell or in an affected cell. The antisense oligonucleotides of the invention preferably comprise between about fourteen and about fifty nucleotides. More preferably, the antisense oligonucleotides comprise between about twelve and about thirty nucleotides. The antisense oligonucleotides of the invention include, but are not limited to, phosphorothioate oligonucleotides and other modifications of oligonucleotides. Methods for synthesizing oligonucleotides, phosphorothioate oligonucleotides, and otherwise modified oligonucleotides are well known in the art (U.S. Pat. No. 5,034,506; Nielsen i., Science 254:1497 (1991)).

Generally, “antisense” RNA sequences are complementary to all or a part of the coding sequence of an mRNA, although there may be some “mismatch” so long as the antisense RNA hybridizes with and inhibits translation of the mRNA. Small interfering RNA (siRNA) are generally short (e.g., 21-23 nucleotides long) double stranded RNA (dsRNA) containing 1-2 nucleotide 3′ overhangs. siRNA facilitates the cleavage and degradation of its complementary mRNA.

Antisense molecules and oligonucleotides of the invention may include those which contain intersugar backbone linkages, such as phosphotriesters, methyl phosphonates, short chain alkyl or cycloalkyl intersugar linkages or short chain heteroatomic or heterocyclic intersugar linkages, phosphorothioates and those with CH₂—NH—O—CH₂, CH₂—N(CH₃)—O—CH₂ (known as methylene(methylimino) or MMI backbone), CH₂—O—N(CH₃)—CH₂, CH₂—N(CH₃)—N(CH₃)—CH₂ and O—N(CH₃)—CH₂—CH₂ backbones (where phosphodiester is O—P—O—CH₂). Oligonucleotides having morpholino backbone structures may also be used (U.S. Pat. No. 5,034,506). In alternative embodiments, antisense oligonucleotides may have a peptide nucleic acid (PNA, sometimes referred to as “protein nucleic acid”) backbone, in which the phosphodiester backbone of the oligonucleotide may be replaced with a polyamide backbone wherein nucleosidic bases are bound directly or indirectly to aza nitrogen atoms or methylene groups in the polyamide backbone (Nielsen et al., Science 254:1497 (1991) and U.S. Pat. No. 5,539,082). The phosphodiester bonds may be substituted with structures which are chiral and enantiomerically specific. Persons of ordinary skill in the art will be able to select other linkages for use in practice of the invention.

Oligonucleotides may also include species which include at least one modified nucleotide base. Thus, purines and pyrimidines other than those normally found in nature may be used. Similarly, modifications on the pentofuranosyl portion of the nucleotide subunits may also be effected. Examples of such modifications are 2′-O-alkyl- and 2′-halogen-substituted nucleotides. Some specific examples of modifications at the 2′ position of sugar moieties which are useful in the present invention are OH, SH, SCH₃, F, OCN, O(CH₂)ₙ, NH₂ or O(CH₂)ₙCH₃ where n is from 1 to about 10; C₁ to C₁₀ lower alkyl, substituted lower alkyl, alkaryl or aralkyl; C₁; Br; CN; CF₃; OCF₃; O-, S-, or N-alkyl; O-, S-, or N-alkenyl; SOCH₃; SO₂ CH₃; ONO₂; NO₂; N₃; NH₂; heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. One or more pentofuranosyl groups may be replaced by another sugar, by a sugar mimic, such as cyclobutyl or by another moiety which takes the place of the sugar.

Controlled release of encapsulant. The exemplified polymersomes provide controlled release through a blend ratio (mol %) of hydrolysable PEO-block copolymer of the hydrophilic component(s) and of the more hydrophobic block copolymer component(s) to produce amphiphilic high molecular weight PEO-based polymersomes, wherein the PEO volume fraction (f_{EO}) and chain chemistry control encapsulant release kinetics from the copolymer vesicles and the polymersome carrier membrane destabilization.

The polymersome membrane can exchange material with the “bulk,” i.e., the solution surrounding the vesicles. Each component in the bulk has a partition coefficient, meaning it has a certain probability of staying in the bulk, as well as a probability of remaining in the membrane. Conditions can be predetermined so that the partition coefficient of a selected type of molecule will be much higher within a vesicle's membrane, thereby permitting the polymersome to decrease the concentration of a molecule, such as cholesterol, in the bulk. In a preferred embodiment, phospholipid molecules have been shown to incorporate within polymersome membranes by the simple addition of the phospholipid molecules to the bulk. In the alternative, polymersomes can be formed with a selected molecule, such as a hormone, protein, oligonucleotide, gene, or the like incorporated within the membrane, so that by controlling the partition coefficient, the molecule will be released into the bulk when the polymersome arrives at a destination having a higher partition coefficient.

Polymersomes of the present invention are particularly useful for the transport of active agents, e.g., antisense oligonucleotides (AON) and the like, but the key to their effectiveness is combining the block copolymers in a manner that provides a method for controlling the release of the encapsulated active agent at a time and location where the released composition is most useful, for example, within a cell target. In addition, the PEO polymersome vesicles of the current invention are ideal for nuclear delivery of encapsulated molecules because they are biocompatible; that is they contain no organic solvent residue and are made of nontoxic materials that are compatible with biological cells and tissues. Thus, because they can interact with plant or animal tissues without deleterious immunological effects, any active agent or molecule deliverable to a patient could be incorporated into a biocompatible polymersome for delivery.

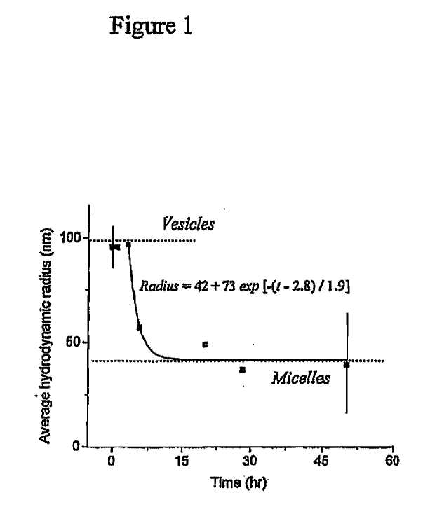

Polymersomes of the present invention are degradable, meaning that, upon uptake of polymersome vesicles by endolysosomes, the membrane of the polymersome begins to degrade as amphiphilic copolymers undergo hydrolysis. Structural changes during degradation as encapsulant is released from polymersomes may be assessed by methods, such as Dynamic Light Scattering. FIG. 1 shows that exemplary degradable polymersomes of the present invention transform to small, surfactant-like micelles just after releasing encapsulated antisense oligonucleotides (AON). High concentrations of such micelles within small endolysosomes within a cell will tend to lyse the endolysosomes, and thus, foster release of encapsulated AON inside the cell, thereby facilitating nuclear delivery of AON.

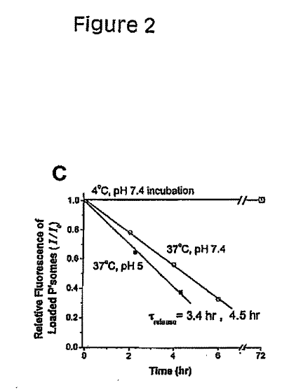

FIG. 2 shows that degradation of exemplary AON-encapsulated polymersomes leads to release of AON from polymersomes. Neutral, nano-transforming polymersomes are capable of delivering encapsulated nucleic acids, such as antisense RNA, into a cell where the released encapsulant is taken up and localized within the cell nucleus (described in more detail in Example 1).

Because polymersomes are exceptional vehicles for the controlled delivery and release of encapsulated active agents into a nucleus of a cell target, encapsulated polymersomes according to the present invention are especially suited for molecular therapies for treating patients suffering from disorders with a genetic and/or molecular basis. For example, disorders, such as Duchenne Muscular Dystrophy and other molecular-based disorders, such as cancers, including those induced by carcinogens, viruses and/or dysregulation of oncogene expression.

Dosages for a given encapsulated polymersome can be determined using conventional considerations, e.g., by customary comparison of the differential activities of the subject preparations and a known appropriate, conventional pharmacological protocol. Dosages further depend on route of administration. The appropriate administration route and dosage vary in accordance with various parameters, for example with the individual being treated or the disorder to be treated, or alternatively with the therapeutic active agents or gene(s) of interest to be transferred. The particular formulation employed will be selected according to conventional knowledge depending on the properties of the tumor, or hyperproliferative target tissue and the desired site of action to ensure optimal activity of the active ingredients, i.e., the extent to which the encapsulated active agent reaches its target tissue following delivery by the methods and system herein.

Polymersomes encapsulated with nucleic acids, such as antisense oligonucleotides, have many promising therapeutic applications. Polymersomes of the present invention are biocompatible and can be used to deliver nucleic material to cells to correct errors in protein expression, or to inhibit gene expression (gene silencing), or could be used in combination with traditional therapies, such as drug therapy, for patients suffering from diseases with a molecular basis, such as cancer. Combination therapy is also a promising approach to cancer treatment, where siRNA-encapsulated polymersomes and anticancer drugs working in concert may overcome the drug resistance often seen in cancer patients, as well as enhance treatments of chemotherapy.

The present invention is further described in the following examples. These examples are not to be construed as limiting the scope of the appended claims.

### EXAMPLES

### Example 1

### Nuclear Delivery of Antisense Oligonucleotide (AON) by Degradable Controlled-Release Neutral Polymersomes In Vitro and In Vivo

Copolymers used in this study are listed in Table 1. PEG-polycaprolactone (PEG-PCL) was from Polymersource (Montreal, Canada) and further purified as needed. PEG-polybutadiene (PEG-PBD) block copolymers were synthesized by anionic polymerization. Dialysis tubing was purchased from Spectrum (Rancho Dominguez, Calif.). Chloroform was from Fisher Scientific (Suwanee, Calif.). Absolute alcohol, DMSO, PKH26 and PKH67 cell tracking dye, phosphate buffered saline (PBS) were from Sigma-Aldrich (St. Louis, Mo.). Tetramethyl rhodomine carboxyl azide (TMRCA), fluorescein-5-carbonyl azide and Alexa Fluor anionic dextran were from Molecular Probes (Eugene, Oreg.).

TABLE 1
Properties of degradable and non-degradable
block copolymer amphiphiles.

| | | M.W | Poly | PEG |
|---|---|---|---|---|
| copolymer | Aₘ-Bₙ | (kg/mol) | dispersity | weight frac. |
| PEG-PCL | EO₅₂-CL₄₄ | 7.0 | 1.30 | 0.29 |
| PEG-PBD | EO₂₆-BD₄₆ | 3.6 | 1.09 | 0.33 |

Polymer vesicle preparation. PEG-PCL blended with inert PEG-PBD copolymers provides broad control over release kinetics from polymersomes. Degradable polymersomes used here were composed of 25/75% (PEG-PBD and PEG-PCL), prepared by mixing of copolymers (0.2 to 5.0 mg/ml) dissolved in DMSO with PBS solution (15:85 v/v). For tracking copolymer fate in cells or tissue, hydrophobic fluorophore, TMRCA, was chemically conjugated on the PEG side of PEG-PBD copolymers. The hydroxyl end of the PEG was covalently attached to tetramethyl rhodamine (TMR) through a rearrangement of an acyl azide. Briefly, a TMR acyl azide (Molecular Probes) was heated in toluene at 80° C. to cause rearrangement to an isocyanate. Simultaneously, 0.5 mg of PEG-PBD was added in a molar ratio of 10:1 dye:copolymer for 12 hours; 20 mg of NH₃OH was then added to the stirred solution for 2 hours to de-protect the non-fluorescent urethane derivative, which turned the solution color from pink to a deep red.

AON loading in polymer vesicles. Copolymer solutions were prepared fresh for each use to prevent the hydrolysis of PEG-PCL. At room temperature, copolymer solutions were added slowly to oligonucleotide solutions in deionized water to reach the required concentration and vortexed briefly. DLS measurements showed that the order which the components were added did not influence the particle size distribution and showed that the polymer concentration did not influence the particle size distribution (not shown). The mixed solution was dialyzed (3.5 kDa) in cold PBS to extract the DMSO. To generate 100-nm vesicles, vesicles were extruded through nano-porous filters. The oligonucleotide employed was a 2′O-methyl 20-mer oligoribonucleotide (5′-UCCAUUCGGCUCCAAACCGG-3′) (SEQ ID NO:1). During synthesis (Proligo, Boulder, Colo.) each base was phosphorothioated and contained a methoxy group at the 2′ carbon. A 6-FAM moiety (fluorescein isothiocyanate [FITC] derivative) was covalently linked to the 5′ end of the AON. The phosphorothioation and 2′-O-methylation have been previously shown to reduce nuclease degradation and to increase hybridization to the target pre-mRNA 14. After 24 hr dialysis at 4° C. to remove free AON, the loaded vesicles were used for cell culture and mouse studies. To ensure complete dialysis of free AON from loaded polymersomes, free AON was dialyzed in parallel. For control studies, negatively charged dextran of similar molecular weight (10 kDa) was loaded in vesicles. Hydrophobic fluorescent dyes (PKH26 or PKH67) to label polymersomes if needed were added directly to a vesicle suspension. Stability of AON loaded vesicles was evaluated as a function of pH (5.5 and 7.4) and temperature (4° C. and 37° C.).

Visualization & Fluorescence measurements. Vesicles were imaged with an Olympus IX71 inverted fluorescence microscope with a 60× oil objective and a Cascade CCD camera. The hydrophobic fluorescent drugs/dyes that have partitioned into the bilayer membrane cores allow the imaging of vesicles with diameters >1 μm. TMRCA conjugated copolymer enabled the imaging of vesicles with diameters >0.5 μm. Cultured cells were imaged at 20×, 40× and 60× magnifications. Photo bleaching studies were conducted using a pulsed dye laser (Photonic Instruments, St. Charles, Ill.), on a NIKON TE300 inverted fluorescence microscope, imaged with a 60× oil immersion objective. Image J Freeware (Java-based Digital Imaging and Communications in Medicine (DICOM) viewer) (NIH) was used for image analysis. Fluorescence images were used to measure the change of fluorescence intensity over time and the morphology of polymer vesicles, cultured myotubes and muscle sections from mouse studies. The fluorescent intensity of AON and tetramethylrhodamine-5 carbonyl azide (TMRCA)-tagged vesicles was measured with spectrofluorimetry. The excitation/emission maxima of AON are 492/520 nm and those of TMRCA-tagged vesicles are 545/578 nm.

C2C12 Cell culture. To assess such uptake of polymersome-AON, mouse-derived C2C12 cells were grown on micro-patterned collagen strips (Millipore, Billerica, Mass.). Cells were differentiated to obtain myotubes; allowing a sparse monolayer of well-separated myotubes (mature muscles cells) for clear visualization.

C2C12 murine skeletal myocytes (CRL-1772 from ATCC, Rockville, Md.) were maintained in 75-cm2 flasks (Corning Glass Works, Corning, N.Y.) in 10 mL DMEM supplemented with 20% fetal bovine serum, 0.5% chick embryo extract, and 0.5% penicillin/streptomycin (10,000 units/mL and 10,000 mg/mL, respectively); all culture reagents from GIBCO (Grand Island, N.Y.). Cells were passaged every 2-3 days. In preparation for the experiment, micropatterned slides or collagen-coated 6-well tissue culture petri dishes were seeded with cells. One day after plating, the media was changed to differentiation media (DMEM supplemented with 10% horse serum and 0.5% penicillin/streptomycin). The cells were differentiated for 10 days to obtain mature myotubes and DM medium was replaced every alternate day.

Polymersome delivery into cultured cells. AON-loaded polymersomes were added at 1 mg/ml copolymer and 2 μg/ml AON concentrations to cultured C2C12 myotubes. After 3 h of incubation the medium was replaced with fresh DMEM medium. Saturation in vesicle uptake occurred within hours, with a time constant of 1.5 hrs and sustained perinuclear localization for at least 4 days. Punctate, internalized vesicles appeared distributed throughout the cells with the accumulation of polymers in the perinuclear regions being consistent with vesicle localization in endolysosomes. Polymersome internalization and AON delivery to myotube nuclei was detected by fluorescence microscopy after staining with 4′-6-diamidino-2-phenylindole (DAPI) (Sigma). DAPI is known to form fluorescent complexes with natural double-stranded DNA. Constant sensitivity and gain were maintained for fluorescence intensity analysis, and images were analyzed on a 16 bit scale.

High magnification images of AON-labeled nuclei indicate broadly diffuse and non-specific interactions of AON throughout the nucleoplasm, and also generally showed between 3 and 20 (>50 myotubes) bright “nuclear bodies” of localized AON per field of view. To clarify the nature of the interactions of the diffuse and localized pools, the mobility of AON was assessed by fluorescence recovery after photobleach (FRAP) methods, using a pulsed dye laser for rapid bleaching. With the diffuse AON, FRAP shows essentially complete recovery of fluorescence within about 5 sec, with t_{1/2}˜2 sec, where t_{1/2} is the time required for the bleach spot to recover half of its initial intensity, indicating high mobility. In contrast, recovery after FRAP of AON within the nuclear bodies is minimal and indicates strong binding in the nuclear body. Since base pairing interactions are generally temperature dependent, diffusion was compared at 22° C. to 42° C., but no difference could be measured. FRAP studies clearly establish dynamic localization within the nucleoplasm, which is at least consistent with the need for on-and-off splicing of pre-mRNA to effect function.

Intramuscular injection in mdx-mice. The dystrophin-deficient mdx mouse is a widely used animal model for muscular dystrophy. Additionally, intramuscular injections test principles of delivery separate from issues of in vivo circulation; free, unencapsulated AON does not circulate more than a few minutes following systemic injection whereas polymersomes circulate for hours.

Tibialis anterior (TA) muscles of mdx mice (6-8 wks of age) were injected at mid-muscle with a 30 μl solution of either free AON (control) or AON-polymersome (5.0 μg AON and 1.5 mg/ml polymer concentration). Post-injection, the mice (duplicates) were divided in two groups. One group was sacrificed 12 hrs later to study AON nuclear delivery and the latter group was sacrificed after 3 weeks for dystrophin expression. Briefly, TA muscles were snap frozen in OCT medium (Gibco) and stored at −70° C. Approximately 50 cryo-sections (of 7 μm each) were obtained to cover the entire length of each TA muscle. The sections were fixed in methanol for 1 min, blocked and immunostained for dystrophin using Dys1 and Dys2 antibodies (Novacastra, Newcastle, UK) at 1:100 dilutions. These were incubated at 4° C. overnight, the slides were washed three times with PBS and then further incubated for 1 hr with secondary antibodies (1:1000). After washing with PBS and Hoechst staining, the slides were mounted using gel-mount (Biomedia; Sigma). Nuclear uptake of fluorescent AON was evaluated by fluorescence imaging (20× or 60× objectives) of DAPI stained nuclei. For each sample, more than 10,000 nuclei were counted from randomly selected fields. Dystrophin-positive fibers were counted using Image J freeware and compared to control, mid, and end-sections of TA muscle. To quantify dystrophin expression, more than 4000 fibers were counted from randomly selected fields.

Following injection, nuclear localization of polymersome-delivered AON was readily apparent in TA muscle within 12 hrs, shown by fluorescent images. Fluorescent dyes included red, green, and Hoechst-blue indicator dyes for visualizing localization of AON within the cell by fluorescent microscopy. Fluorescent copolymer (labeled in red—not shown here) showed a diffuse distribution compared to green-AON, and free-unencapsulated-AON showed relatively little evidence of nuclear localization. Delivery efficiency was quantified by simply counting the number of green-AON-nuclei and dividing by the number of Hoechst-labeled (a stain specific for cell nuclei) blue nuclei. AON-polymersomes gave a mean delivery efficiency of over 50% and showed a relatively even distribution along the entire muscle length. In contrast, free AON showed less than 10% efficiency and appeared primarily localized to the nuclei of mid-section muscle in close proximity to the injection site.

Three weeks after a single intramuscular injection of the AON polymersome formulations into mdx mice, dystrophin expression was directly visualized by immunostaining. To confirm that AON delivered successfully skips the defective exon 23 in the mdx mouse, expression was evaluated for a protein that is only 71 amino acids shorter than full-length dystrophin. Immuno detection of dystrophin protein was done with Dys1 antibody to the N-terminus and with Dys2, which is a C-terminal specific antibody that will detect only the corrected dystrophin protein.

Dystrophin expression post-AON delivery with polymersomes proved robust in clearly showing a membrane localization pattern similar to that of normal muscle, when viewed at 20× magnification following dystrophin immunostaining with dystrophin antibodies. Widespread, membrane-localized dystrophin expression was observed not only across the muscle mid-section, but also toward the ends of the muscles. Muscle sections from mid-section to the end were imaged and muscle fibers observed to count the dystrophin-positive fibers. By counting more than 4500 muscle fibers, dystrophin-positive fibers induced by polymersome-AON were 26% and the control sample was no more than 6%, thus yielding a 4.3-fold increase in dystrophin expression with AON-encapsulated polymersomes. In comparison, muscle sections injected with empty vesicles showed zero expression.

The broad distribution of expression highlights the substantial perfusion of these stealthy, controlled release vesicle carriers along the entire muscle length. The distal expression offered clear evidence of transport of AON in polymersomes that is simply not seen with free AON. The nontoxic nature of the copolymer is also evident in the fact that no obvious degeneration was observed in polymer treated mdx muscles compared to controls. Overall, the identified strong dystrophin expression profile confirmed AON localization to myonuclei.

### Example 2

### Cellular Delivery of siRNA by PEO-PLA Bilayer Polymersomes

To determine if polymersomes are efficient nano-delivery systems for enabling gene silencing by siRNA, PEO-based polymersomes were independently encapsulated with two small interfering RNAs; siRNA for clusterin, and siRNA for lamin A/C.

The lamin family of proteins make up the nuclear lamina, a matrix of protein located next to the inner nuclear membrane (also known as LMNA). Lamin proteins are involved in nuclear stability, chromatin structure and gene expression. There are two types of mammalian lamin, A and B. Through alternate splicing, this gene encodes three type A lamin isoforms. Mutations in the lamin A/C gene lead to a number of diseases: Emery-Dreifuss muscular dystrophy type 2, familial partial lipodystrophy, limb girdle muscular dystrophy type 1B, dilated cardiomyopathy, familial partial lipodystrophy, Charcot-Marie-Tooth disorder type 2B1, mandibuloacral dysplasia, childhood progeria syndrome (Hutchinson-Gilford syndrome) and a subset of Werner syndrome. These diseases have, therefore, been referred to as laminopathies.

Using PEO-PLA polymersomes are of bilayer vesicular structure synthesized from amphiphilic polymers (f_{EO}<˜0.28) by the film hydration method, the resulting liposome-like structures were completely PEGylated to avoid clearance by the immune system during circulation. Poly (ethylene oxide)-poly (lactic acid) (PEO 0.7 kDa-PLA 5 kDa) was from Polymersource, Inc.

Using fluorescently labeled siRNA, the time course of gene silencing was examined at both the mRNA and protein levels. Fluorescein isothiocyanate (FITC) was conjugated to siRNA, resulting in FITC-labeled siRNA against lamin A/C. (Conjugation kits and conjugated siRNA are commercially available from Dharmacon, Lafayette, Colo.) Lipofectamine was purchased from Invitrogen, Inc. and the lamin A/C fluorescence immunoassay kit was purchased from Roche, Inc.

Preparation of siRNA encapsulated polymersomes. The encapsulation procedure was similar to the method described in Example 1. In brief, 0.1 ml of FITC-labeled-siRNA (300 ug/ml) was added to the PEO-PLA polymersome solution in DMSO (2 mg/ml) (Gibco) and mixed for 15 seconds. The mixture was added to 3.9 ml of H₂O to make a 5 ml suspension. The suspension was transferred to a dialysis cassette (10,000 MWCO) and dialyzed against water for 4 hrs to remove DMSO. Dialysis continued overnight with dialysis tubing (300,000 MWCO) to remove unencapsulated siRNA. The encapsulation of siRNA was verified with a fluorescence microscope and encapsulation efficiency was determined by fluorospectrometer.

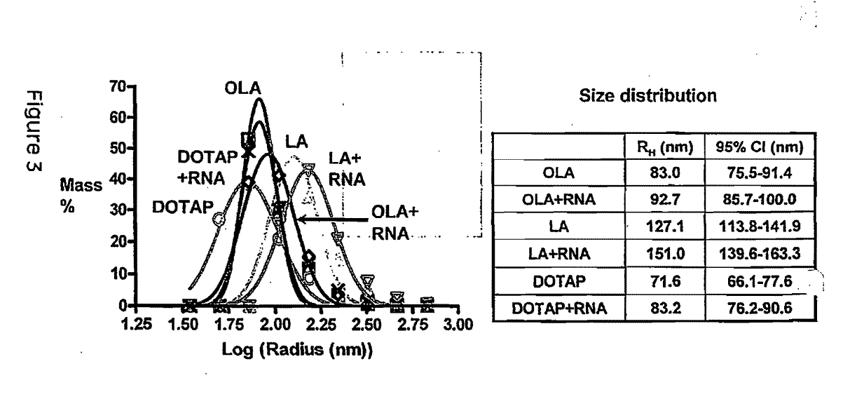

FIG. 3 shows the hydrodynamic size distribution of PEO-PLA polymersomes, with and without encapsulated material, as well as comparison with commercially available transfection controls (LA). Encapsulation of siRNA (15 kDa) slightly increased the particle size. PEO-PLA polymersomes alone measure 83 nm radially, whereas the encapsulation of siRNA increased the polymersome radius to 92.7 nm.

Transfection of siRNA loaded polymersome and lipofectamine to A549 cells. Encapsulation of FITC-siRNA against lamin A/C into lipofectamine (LA) was according to transfection reagent protocol. Both PEO-PLA polymersome and LA loaded with siRNA against lamin A/C were transfected to human lung carcinoma A549 cells (ATCC, Manassas, Va.) by incubating various concentrations of the gene carriers with 50,000 cells/well in a 24-well plate at 37° C. for 6 hrs. After the incubation, the medium was replaced with fresh medium and the incubation continued for 3 days. Both LA-RNA and PEO-PLA polymersome encapsulating siRNA were internalized by cells, as viewed by microscopy.

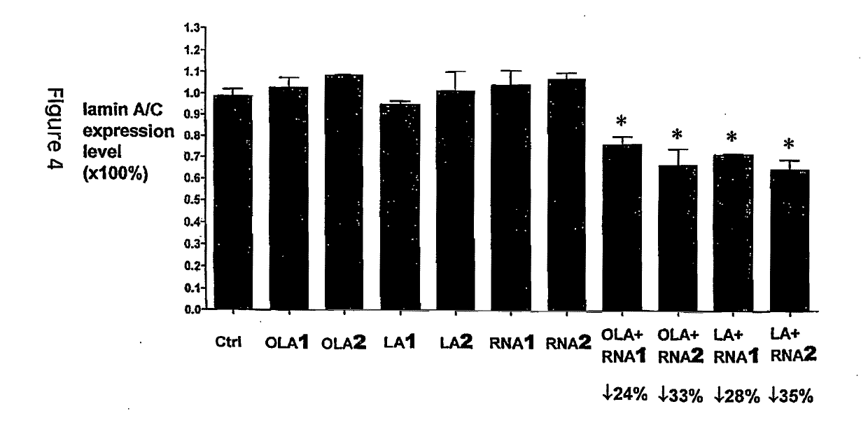

Lamin A/C gene silencing efficiency was determined by measuring the lamin expression level with fluorescence-immunoassay. In 24-well plates (50,000 cells/well) siRNA encapsulated polymersomes were incubated with cells at two doses: 125 ng/17 nM and 250 ng/33 nM. After 72 hours, lamin A/C gene expression was reduced by 24% at dose one and 33% at dose two. (See FIG. 4) Lamin A/C expression was also measured following 96 hours of incubation of cells with siRNA encapsulated PEO-PLA polymersomes at a dose of 125 ng/33 nM. Lamin A/C expression was reduced by 26% compared to controls. (See FIG. 6).

PEO-PLA polymersomes encapsulated with siRNA against lamin A/C successfully delivered siRNA into cells and achieved biological effects in comparable efficiencies to other gene carriers. Separately, but not shown, PEO-based polymersomes were encapsulated with siRNA against clusterin, which is overexpressed in lung cancer and contributes to drug resistance often seen in cancer patients undergoing treatment. Clusterin is an 80 KDa protein encoded by a gene located on chromosome 8. It is highly conserved across species and shows wide tissue distribution. It is implicated in a variety of activities, such as programmed cell death, regulation of complement mediated cell lysis, membrane recycling, cell-cell adhesion and src induced transformation. Overexpression of clusterin was reduced by gene silencing using siRNA encapsulated PEO-polymersomes (data not shown here). Thus, PEO based polymersomes provide a novel and useful treatment for cancer or in combination therapy with anti-cancer drugs or conventional chemotherapy.

Each and every patent, patent application and publication that is cited in the foregoing specification is herein incorporated by reference in its entirety.

While the foregoing specification has been described with regard to certain preferred embodiments, and many details have been set forth for the purpose of illustration, it will be apparent to those skilled in the art that the invention may be subject to various modifications and additional embodiments, and that certain of the details described herein can be varied considerably without departing from the spirit and scope of the invention. Such modifications, equivalent variations and additional embodiments are also intended to fall within the scope of the appended claims.

1. A method for delivering an oligonucleotide, including antisense RNA, siRNA and RNAi, to a cell or tissue target, the method comprising:
encapsulating the oligonucleotide in a biodegradable neutral, nano-transforming polymersome delivery vehicle; and
delivering the encapsulated oligonucleotide to the cell or tissue target in vitro or in vivo, wherein the polymersome delivery vehicle decomposes at a known rate at a known pH, thereby releasing the encapsulated oligonucleotide within the cell or tissue target in a controlled manner.

2. The method of claim 1, wherein delivering comprises delivery to a cell of a patient in need thereof in vivo.

3. The method of claim 1, wherein the cell or tissue target comprises a muscle cell.

4. The method of claim 1, wherein delivering the encapsulated oligonucleotide comprises delivering in vivo to the targeted cells or tissue in a model animal for muscular dystrophy.

5. The method of claim 1, wherein delivering the encapsulated oligonucleotide comprises delivering in vivo to the targeted cells or tissue in a human muscular dystrophy patient having a need therefor.

6-7. (canceled)

8. A biodegradable polyethylene oxide (PEO)-based polymersome delivery system comprising at least one antisense oligonucleotide encapsulated therein for delivery to the nucleus of a cell or tissue target, wherein the PEO-based polymersome system comprises one or more amphiphilic copolymers that self-assemble in aqueous solution, and wherein at least one of the amphiphilic copolymers is a hydrophilic block copolymer, the weight fraction of which, relative to total copolymer molecular weight, directs self-assembly of the amphiphilic molecules into a bilayer vesicular structure having a neutral surface charge, and wherein the PEO volume fraction and chain chemistry control antisense oligonucleotide release kinetics thereby regulating polymersome membrane degradation.

9. The PEO-based polymersome system of claim 8, wherein the antisense oligonucleotide comprises antisense RNA, siRNA or RNAi.

10. The PEO-based polymersome system of claim 8, wherein the hydrophilic copolymer comprises polyethelyne oxide.

11. The PEO-based polymersome system of claim 8, wherein the hydrophobic copolymer comprises polylactide or polycaprolactone.

12. The PEO-based polymersome system of claim 8, wherein the polymersome system and the encapsulant are each biocompatible.

13. The PEO-based polymersome system of claim 8, wherein the polymersome vesicle facilitates nuclear delivery of encapsulated oligonucleotide within a cell in vivo.

14. The PEO-based polymersome system of claim 8, wherein degradation of the biodegradable vesicle occurs within cellular endolysosomes, thereby fostering release of encapsulated oligonucleotide and presentation of oligonucleotide to the nucleus of the cell.

15. The method of claim 2, wherein the method of delivering the encapsulated nucleotide further comprises delivering the nucleotide to the nucleus of the muscle cell, effecting protein expression along the length of the muscle and production of dystrophin positive fibers.

16. The method of claim 3, wherein the method of delivering the encapsulated nucleotide further comprises delivering the nucleotide to the nucleus of the muscle cell, effecting protein expression along the length of the muscle and production of dystrophin positive fibers.

17. The method of claim 1, wherein the decomposing of the polymersome vehicle occurs within endolysosomes, thereby fostering release of encapsulated oligonucleotide.

18. The method of claim 2, wherein the decomposing of the polymersome vehicle occurs within endolysosomes, thereby fostering release of encapsulated oligonucleotide.

19. The method of claim 3, wherein the decomposing of the polymersome vehicle occurs within endolysosomes, thereby fostering release of encapsulated oligonucleotide.

20. The method of claim 4, wherein the decomposing of the polymersome vehicle occurs within endolysosomes, thereby fostering release of encapsulated oligonucleotide.

21. The method of claim 5, wherein the decomposing of the polymersome vehicle occurs within endolysosomes, thereby fostering release of encapsulated oligonucleotide.

22. The method of claim 15, wherein the decomposing of the polymersome vehicle occurs within endolysosomes, thereby fostering release of encapsulated oligonucleotide.

23. The method of claim 16, wherein the decomposing of the polymersome vehicle occurs within endolysosomes, thereby fostering release of encapsulated oligonucleotide.
